(19) United States
(12) Patent Application Publication
(10) Pub. No.: US 2022/0338739 A1
(43) Pub. Date: Oct. 27, 2022

(54) SYSTEMS, DEVICES, AND METHODS FOR PERFORMING TRANS-ABDOMINAL FETAL OXIMETRY AND/OR TRANS-ABDOMINAL FETAL PULSE OXIMETRY USING AN ACOUSTIC AND/OR ACOUSTO-OPTICAL SIGNAL

(71) Applicant: RAYDIANT OXIMETRY, INC., San Ramon, CA (US)

(72) Inventors: JANA M. KAINERSTORFER, Pittsburgh, PA (US); MARK ANDREW ROSEN, Piedmont, CA (US); NEIL PADHARIA RAY, Sacramento, CA (US)

(21) Appl. No.: 17/761,069

(22) PCT Filed: Sep. 16, 2020
(86) PCT No.: PCT/US20/70543

§ 371 Date: Mar. 16, 2022

Related U.S. Application Data
(60) Provisional application No. 62/900,853, filed on Sep. 16, 2019.

(51) Int. Cl. A61B 5/00; A61B 5/1455; A61B 8/08; A61B 8/02

(52) U.S. Cl. CPC A61B 5/0095; A61B 5/14551; A61B 8/488; A61B 8/02

(57) ABSTRACT

Photoacoustic and/or acousto-optical techniques may be used to transabdominally perform fetal oximetry and/or trans-abdominal fetal pulse oximetry. In some cases, a composite acoustic signal that has emanated from an abdomen of a pregnant mammal may be received by a processor from, for example, an ultrasonic detector and/or microphone positioned on, or near, a pregnant mammal's abdomen and the composite acoustic signal may result from an optical signal incident on the pregnant mammal's abdomen and a fetus contained therein. A portion of the composite acoustic signal that was incident on the fetus may be isolated from the composite acoustic signal and then analyzed to determine a fetal hemoglobin oxygen saturation level and/or a fetal tissue oxygen saturation level.

### RELATED APPLICATION

This application is a NON-PROVISIONAL application of U.S. Provisional Patent Application No. 62/900,853 filed on 16 Sep. 2019 entitled “SYSTEMS, DEVICES, AND METHODS FOR PERFORMING TRANS-ABDOMINAL FETAL OXIMETRY AND/OR TRANS-ABDOMINAL FETAL PULSE OXIMETRY USING PHOTOACOUSTIC INFORMATION,” which is incorporated herein by reference in their respective entireties.

### FIELD

The present disclosure relates to medical devices and, in particular, to medical devices used to transabdominally image a fetus, determine a level of fetal hemoglobin oxygenation, and/or fetal tissue oxygenation using photoacoustic and/or acousto-optic imaging techniques.

### BACKGROUND

Photoacoustic imaging may employ the photoacoustic effect to image biological tissue (e.g., muscle, fat, brain, etc.) and fluids (e.g., blood) in a body. Often times, a non-ionizing laser is used to deliver an optical signal to biological tissue. Some of the optical energy may be absorbed by the tissue and converted into heat. This conversion can result in transient thermoelastic expansion which causes the tissue to emit a sonic signal, usually in the ultrasonic frequency range. The sonic signal may be detected by, for example, ultrasonic transducers and/or microphones and analyzed to determine properties of the tissue.

Acousto-optical technology pertains to the interaction between sound waves and light waves wherein both sound waves and a light waves may be directed toward biological tissue and an interaction between the two may be analyzed to determine a property of the biological tissue.

### SUMMARY

Systems, devices, and methods for performing trans-abdominal fetal oximetry and/or trans-abdominal fetal pulse oximetry using photoacoustic and/or acousto-optical techniques are herein disclosed. In some embodiments, a composite acoustic signal that has emanated from an abdomen of a pregnant mammal may be received by a processor from, for example, an ultrasonic detector and/or microphone. The composite acoustic signal may result from an optical signal incident on the pregnant mammal's abdomen and a fetus contained therein. In some instances, the optical signal may be projected by a laser or LED into the pregnant mammal's abdomen and the composite acoustic signal may be caused by transient thermoelastic expansion resultant from an interaction of the pregnant mammal's abdominal tissue and/or the fetus' tissue to the optical signal. At times, the processor also controls the operation of the source of the optical signal.

The composite acoustic signal may then be processed to, for example, isolate a portion of the composite acoustic signal that was incident on the fetus thereby generating a fetal acoustic signal. The isolation may comprise removal of noise and/or confounding effects caused by the pregnant mammal. In some embodiments, the composite acoustic signal may be pre-processed prior to processing the composite acoustic signal to isolate a portion of the composite acoustic signal in order to remove noise from the composite acoustic signal. Noise may be caused by, for example, hum or ambient light. in some embodiments, additional information such as, for example, a fetal depth, a maternal heart rate, a fetal heart rate, a maternal hemoglobin oxygenation level, and/or a maternal acoustic signal may be received prior to processing the composite acoustic signal to generate the fetal acoustic signal and the isolation of the fetal signal may include processing of the composite acoustic signal using the additional information to isolate a portion of the composite acoustic signal contributed by the fetus (i.e., generation of the fetal signal). For example, in some embodiments, a maternal heart rate signal may be received, and, at times, both the composite acoustic signal and maternal heart rate signal may include one or more timestamp. The maternal heart rate signal and the composite acoustic signal may be synchronized in time using the timestamp. In these embodiments, the processing of the composite acoustic signal to isolate a portion of the composite acoustic signal to remove noise from the composite acoustic signal may include determining a portion of the composite acoustic signal that corresponds with the maternal heart rate signal and subtracting the portion of the composite acoustic signal that corresponds to the maternal heart rate signal from the composite acoustic signal to generate the fetal signal.

Additionally, or alternatively, a fetal heart rate signal may be received by the processor. At times, both the composite acoustic signal and fetal heart rate signal may include a timestamp, which may be used to synchronize the fetal heart rate signal and the composite acoustic signal in time. When the fetal heart rate signal is received, the processing of the composite acoustic signal to isolate a portion of the composite acoustic signal to remove noise from the composite acoustic signal may include determining a portion of the composite acoustic signal that corresponds with the fetal heart rate signal and amplifying and/or extracting the portion of the composite acoustic signal that corresponds to the fetal heart rate signal from the composite acoustic signal.

Additionally, or alternatively, a fetal depth (e.g., a distance between the epidermis of the pregnant mammal and the epidermis of the fetus) for the fetus within the pregnant mammal's abdomen may be received. Then, a duration of an interval of time it takes for sound to travel the length of the fetal depth to a detector may be determined. The processing of the composite acoustic signal to isolate a portion of the composite acoustic signal may then include portions of the composite acoustic signal that occur during the interval of time. For example, if it is determined that sound travels the fetal distance in 0.1 seconds, then portions of the composite acoustic signal received in the first 0.1 seconds may be filtered from the composite acoustic signal since they could not have been incident on the fetus.

Additionally, or alternatively, a maternal acoustic signal may be received and, in these cases, isolation of the fetal acoustic signal may include determining a portion of the composite acoustic signal that corresponds with the maternal acoustic signal and subtracting the portion of the composite acoustic signal that corresponds to the maternal acoustic signal from the composite acoustic signal.

Additionally, or alternatively, the composite acoustic signal may be timestamped and a timestamped maternal acoustic signal may be received. Then, isolation of the fetal acoustic signal may include synchronizing the timestamped composite acoustic signal and maternal acoustic signal using a timestamp present in the composite acoustic signal and a timestamp present in the maternal acoustic signal, determining a portion of the composite acoustic signal that corresponds with the maternal acoustic signal, and subtracting the portion of the composite acoustic signal that corresponds to the maternal acoustic signal from the composite acoustic signal.

Once the fetal signal is isolated from the composite acoustic signal, the fetal acoustic signal may be analyzed to determine a fetal hemoglobin oxygen saturation level and/or a fetal tissue oxygen saturation level and an indication of the fetal hemoglobin oxygen saturation level and/or fetal tissue oxygen saturation level to a user as, for example, a numerical value (e.g., 52, 52%, etc.), a range of values (e.g., 40-45, 60-65, etc.) and/or as an image that is coded via, for example, shading and/or color to indicate tissue oxygenation levels for the pregnant mammal and fetus.

In some embodiments, the processor may control operation of a source of the optical signal, such as a laser, LED, and/or array of optical sources (e.g., a fiber optic bundle of cables). In these embodiments, an operation of the source of the optical signal may be adjusted responsively to the fetal depth. Exemplary adjustments include, but are not limited to, a frequency/wavelength of light projected into the pregnant mammal's abdomen, a duration of time the light is projected into the pregnant mammal's abdomen, and a length of time between projections of light into the pregnant mammal's abdomen. Additionally, or alternatively, it may be determined whether the composite acoustic signal is of sufficient strength and the operation of the source of the optical signal may be adjusted responsively to a determination that the composite acoustic signal is not of sufficient strength. Additionally, or alternatively, it may be determined whether the fetal acoustic signal is of sufficient strength and the operation of the source of the optical signal may be adjusted responsively to a determination that the composite acoustic signal is not of sufficient strength.

### BRIEF DESCRIPTION OF THE FIGURES

The present invention is illustrated by way of example, and not limitation, in the figures of the accompanying drawings in which:

FIG. 1 is a block diagram illustrating an exemplary system for determining a level of oxygen saturation for fetal hemoglobin and/or whether meconium is present in the amniotic fluid of a pregnant mammal, consistent with some embodiments of the present invention;

FIG. 2A illustrates provides a midsagittal plane view of pregnant mammal's abdomen with fetal probe positioned thereon, consistent with some embodiments of the present invention;

FIG. 2B illustrates provides a midsagittal plane view of pregnant mammal's abdomen with a fetal probe and acousto-optic modulator positioned thereon, consistent with some embodiments of the present invention;

FIG. 3 provides a flowchart illustrating a process for determining a fetus's tissue and/or hemoglobin oxygen saturation using a photoacoustic signal, consistent with some embodiments of the present invention;

FIG. 4 is a flowchart illustrating an exemplary process for isolating a fetal acoustic signal using fetal depth, consistent with some embodiments of the present invention;

FIG. 5 is a flowchart illustrating an exemplary process for isolating a fetal acoustic signal and/or fetal acousto-optical signal using a maternal heart rate signal, consistent with some embodiments of the present invention;

FIG. 6 is a flowchart illustrating an exemplary process for isolating a fetal acoustic signal and/or fetal acousto-optical signal using a fetal heart rate signal, consistent with some embodiments of the present invention;

FIG. 7 is a flowchart illustrating an exemplary process for isolating a fetal acoustic signal using a maternal acoustic signal, consistent with some embodiments of the present invention; and

FIG. 8 provides an exemplary image of a portion of a pregnant mammal's abdomen where a tissue oxygenation for a maternal layer and a fetal layer are provided as an image where a pattern within the image indicates a level of tissue oxygenation for the respective layers of tissue.

Throughout the drawings, the same reference numerals and characters, unless otherwise stated, are used to denote like features, elements, components, or portions of the illustrated embodiments. Moreover, while the subject invention will now be described in detail with reference to the drawings, the description is done in connection with the illustrative embodiments. It is intended that changes and modifications can be made to the described embodiments without departing from the true scope and spirit of the subject invention as defined by the appended claims.

### WRITTEN DESCRIPTION

Described herein are systems, devices, and methods for photoacoustically imaging a fetus, determining a level of fetal hemoglobin oxygenation, and/or determining a level of fetal tissue oxygenation. Photoacoustic imaging may employ the photoacoustic effect to image biological tissue (e.g., muscle, fat, brain, etc.) and fluids (e.g., blood) in a body. For ease of discussion, biological tissue and fluids may be collectively referred to as “tissue” herein. To photoacoustically image tissue, a pulsed laser may project pulses of one or more wavelengths of light into the tissue of interest. When the tissue absorbs some of the light, energy from the absorbed light may be converted into heat, which may cause thermoplastic expansion of the tissue and emission of soundwaves in accordance with the photoacoustic effect. The emitted sound waves are typically within the ultrasonic range. The emitted sound may be detected by, for example, a microphone or ultrasonic detector, and then analyzed to form an image of the tissue under study using, for example, photoacoustic equations.

In some instances, a magnitude of detected sound (e.g., ultrasonic emission) may be proportional to the local energy deposition of the imaged tissue and may provide an indication of optical absorption contrast for different types and/or regions of tissue that may then be used to generate 2-dimensional and/or 3-dimensional images of targeted areas (e.g., a fetus) that may show tissue and/or hemoglobin oxygen saturation levels for the imaged areas.

The oxygen saturation level of a fetus's hemoglobin and/or tissue may be used by trained medical professionals to assess the health of a fetus as well as a level of stress (e.g., risk of fetal acidosis) it may be under during, for example, a labor and delivery process. Typically values of oxygen saturation for fetal blood fall within the range of 30-60% with anything lower than 30% indicating that the fetus may be in distress and/or at risk of fetal acidosis.

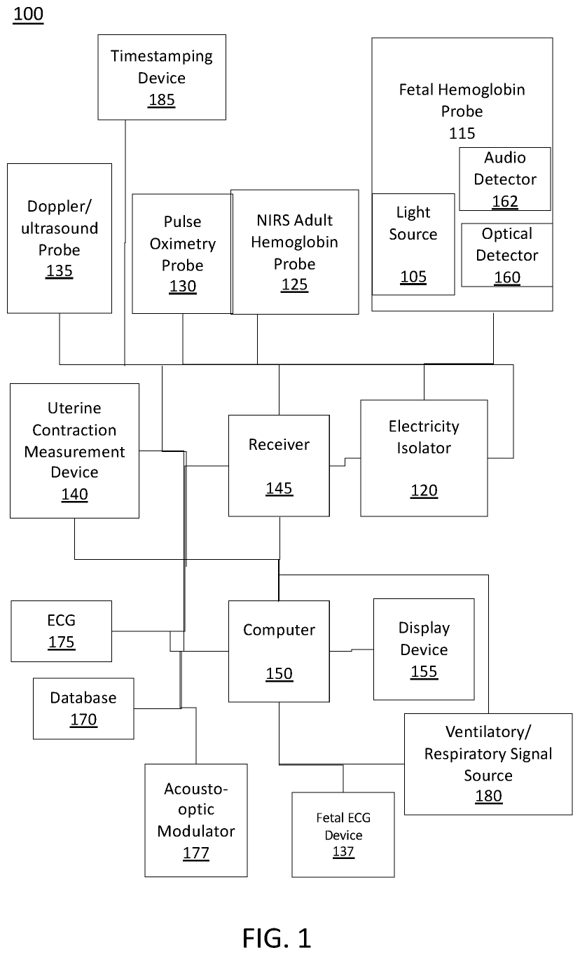

FIG. 1 provides an exemplary system 100 for detecting and/or determining fetal hemoglobin oxygen saturation levels. The components of system 100 may be coupled together via wired and/or wireless communication links. In some instances, wireless communication of one or more components of system 100 may be enabled using short-range wireless communication protocols designed to communicate over relatively short distances (e.g., BLUETOOTH®, near field communication (NFC), radio-frequency identification (RFID), and Wi-Fi) with, for example, a computer or personal electronic device (e.g., tablet computer or smart phone) as described below.

System 100 includes a fetal hemoglobin probe 115 which includes a light source 105, an optical detector 160, and an audio detector 162. Light source 105 may include a single, or multiple light sources and optical detector 160 may include a single, or multiple detectors. Audio detector 162 may be any detector configured to detect an audio, or sound, signal. Exemplary audio detectors 162 include, but are not limited to, microphones and ultrasonic transducers and/or detectors.

Light source(s) 105 may transmit light at light of one or more wavelengths, including light within the red, near infra-red (NIR), and/or infrared bands of the electromagnetic spectrum into the pregnant mammal's abdomen. Typically, the light emitted by light source(s) 105 will be focused or emitted as a narrow beam to reduce spreading of the light upon entry into the pregnant mammal's abdomen. Light source(s) 105 may be, for example, a LED, and/or a LASER (e.g., a pulsed laser), that may in some instances, be coupled to a fiber optic cable. In embodiments where light source 105 is a pulsed laser, the pulsed laser may be configured to emit pulses of light at a repetition rate of, for example, 1-100 MHz. In some instances, the light source(s) 105 may be tunable, or otherwise user configurable, while, in other instances, one or more of the light sources may be configured to emit light within a pre-defined range of wavelengths. Additionally, or alternatively, one or more filters (not shown) and/or polarizers may filter/polarize the light emitted by light source(s) 105 to be of one or more preferred wavelengths. These filters/polarizers may also be tunable or user configurable.

An exemplary light source 105 may have a relatively small form factor and may operate with high efficiency, which may serve to, for example, conserve space and/or weight within fetal probe 115 and/or limit heat emitted by the light source 105. In one embodiment, light source 105 is configured to emit light in the range of 770-850 nm. Exemplary flux ratios for light source(s) include but are not limited to a luminous flux/radiant flux of 175-260 mW/pulse, a total radiant flux of 300-550 mW, and a power rating of 0.6 W-3.5 W.

Optical detector 160 may be configured to detect a light signal emanating (via, for example, reflection, transmission, and/or backscattering) from the pregnant mammal and/or the fetus and convert this light signal into an electronic signal, which may be communicated to a computer, a processor, such as computer 150, and/or an on-board transceiver (not shown) that may be capable of communicating the signal to the computer/processor. This reflected light might then be processed in order to determine how much light, at various wavelengths, is reflected and/or absorbed by the fetal oxyhemoglobin and/or de-oxyhemoglobin so that a fetal hemoglobin oxygen saturation level may be determined.

Exemplary optical detectors 160 include, but are not limited to, cameras, traditional photomultiplier tubes (PMTs), silicon PMTs, avalanche photodiodes, and silicon photodiodes. The light captured by optical detector 160 may be communicated to computer 150 for processing to convert the reflected light into a measurement of fetal hemoglobin oxygen saturation according to, for example, one or more of the processes described herein.

A fetal hemoglobin probe 115 and components therein may be of any appropriate size and, in some circumstances, may be sized to accommodate the size of the pregnant mammal and/or fetus using any appropriate sizing system (e.g., abdomen size and/or fetus age, etc.). Exemplary lengths for a fetal hemoglobin probe 115 include a length of 2 cm-40 cm and a width of 2 cm-10 cm. In some circumstances, the size and/or configuration of a fetal hemoglobin probe 115, or components thereof, may be responsive to skin pigmentation of the pregnant mammal and/or fetus. In some instances, the fetal hemoglobin probe 115 may be applied to the pregnant mammal's skin via tape or a strap that cooperates with a mechanism (e.g., snap, loop, etc.) (not shown).

System 100 includes a number of optional independent sensors/probes designed to monitor various aspects of maternal and/or fetal health, some of which may be in contact with a pregnant mammal. These probes/sensors are a NIRS adult hemoglobin probe 125, a pulse oximetry probe 130, a Doppler and/or ultrasound probe 135, a uterine contraction measurement device 140, a fetal ECG device 137, a ventilatory/respiratory signal source 180, an ECG machine 175, and an acousto-optical modulator 177. Not all embodiments of system 100 will include all of these components and, in some cases, an intrauterine pulse oximetry probe (not shown) may be used to determine the fetus' heart rate.

ECG machine 170 may be used to determine the pregnant mammal's heart rate. The Doppler and/or ultrasound probe 135 may be configured to be placed on the abdomen of the pregnant mammal and may be of a size and shape that approximates a silver U.S. dollar coin and may provide an image of the fetus and/or information regarding fetal position, orientation, and/or heart rate. Pulse oximetry probe 130 may be a conventional pulse oximetry probe placed on pregnant mammal's hand and/or finger to measure the pregnant mammal's hemoglobin oxygen saturation. NIRS adult hemoglobin probe 125 may be placed on, for example, the pregnant mammal's second finger and may be configured to, for example, use near infrared spectroscopy to calculate the ratio of adult oxyhemoglobin to adult de-oxyhemoglobin. NIRS adult hemoglobin probe 125 may also be used to determine the pregnant mammal's heart rate.

Optionally, system 100 may include a uterine contraction measurement device 140 configured to measure the strength and/or timing of the pregnant mammal's uterine contractions. In some embodiments, uterine contractions will be measured by uterine contraction measurement device 140 as a function of pressure (measured in e.g., mmHg) over time. In some instances, the uterine contraction measurement device 140 is and/or includes a tocotransducer, which is an instrument that includes a pressure-sensing area that detects changes in the abdominal contour to measure uterine activity and, in this way, monitors frequency and duration of contractions.

In another embodiment, uterine contraction measurement device 140 may be configured to pass an electrical current through the pregnant mammal and measure changes in the electrical current as the uterus contracts. Additionally, or alternatively, uterine contractions may also be measured via near infrared spectroscopy using, for example, light received/detected by optical detector 160 because uterine contractions, which are muscle contractions, are oscillations of the uterine muscle between a contracted state and a relaxed state. Oxygen consumption of the uterine muscle during both of these stages is different and these differences may be detectable using NIRS.

Measurements and/or signals from NIRS adult hemoglobin probe 125, pulse oximetry probe 130, Doppler and/or ultrasound probe 135, and/or uterine contraction measurement device 140 may be communicated to receiver 145 for communication to computer 150 and display on display device 155 and, in some instances, may be considered secondary signals. As will be discussed below, measurements provided by NIRS adult hemoglobin probe 125, pulse oximetry probe 130, a Doppler and/or ultrasound probe 135, and/or uterine contraction measurement device 140 may be used in conjunction with fetal hemoglobin probe 115 to isolate a fetal pulse signal and/or fetal heart rate from a maternal pulse signal and/or maternal heart rate. Receiver 145 may be configured to receive signals and/or data from one or more components of system 100 including, but not limited to, fetal hemoglobin probe 115, NIRS adult hemoglobin probe 125, pulse oximetry probe 130, Doppler and/or ultrasound probe 135, and/or uterine contraction measurement device 140. Communication of receiver 145 with other components of system may be made using wired or wireless communication.

In some instances, one or more of NIRS adult hemoglobin probe 125, pulse oximetry probe 130, a Doppler and/or ultrasound probe 135, and/or uterine contraction measurement device 140 may include a dedicated display that provides the measurements to, for example, an operator or medical treatment provider. It is important to note that not all of these probes may be used in every instance. For example, when the pregnant mammal is using fetal hemoglobin probe 115 in a setting outside of a hospital or treatment facility (e.g., at home or work) then, some of the probes (e.g., NIRS adult hemoglobin probe 125, pulse oximetry probe 130, a Doppler and/or ultrasound probe 135, uterine contraction measurement device 140) of system 100 may not be used.

In some instances, receiver 145 may be configured to process or pre-process received signals to, for example, make the signals compatible with computer 150 (e.g., convert an optical signal to an electrical signal), improve SNR, amplify a received signal, etc. In some instances, receiver 145 may be resident within and/or a component of computer 150. In some embodiments, computer 150 may amplify or otherwise condition the received reflected signal to, for example, improve the signal-to-noise ratio.

Receiver 145 may communicate received, pre-processed, and/or processed signals to computer 150. Computer 150 may act to process the received signals, as discussed in greater detail below, and facilitate provision of the results to a display device 155. Exemplary computers 150 include desktop and laptop computers, servers, tablet computers, personal electronic devices, mobile devices (e.g., smart phones), and the like. Exemplary display devices 155 are computer monitors, tablet computer devices, and displays provided by one or more of the components of system 100. In some instances, display device 155 may be resident in receiver 145 and/or computer 150.

In some embodiments, a pregnant mammal may be electrically insulated from one or more components of system 100 by, for example, an electricity isolator 120. Exemplary electricity insulators 120 include circuit breakers, ground fault switches, and fuses. In some embodiments, system 100 may include a fetal ECG device 137 configured to provide a fetal heart rate signal to, for example, computer 150 and/or receiver 145.

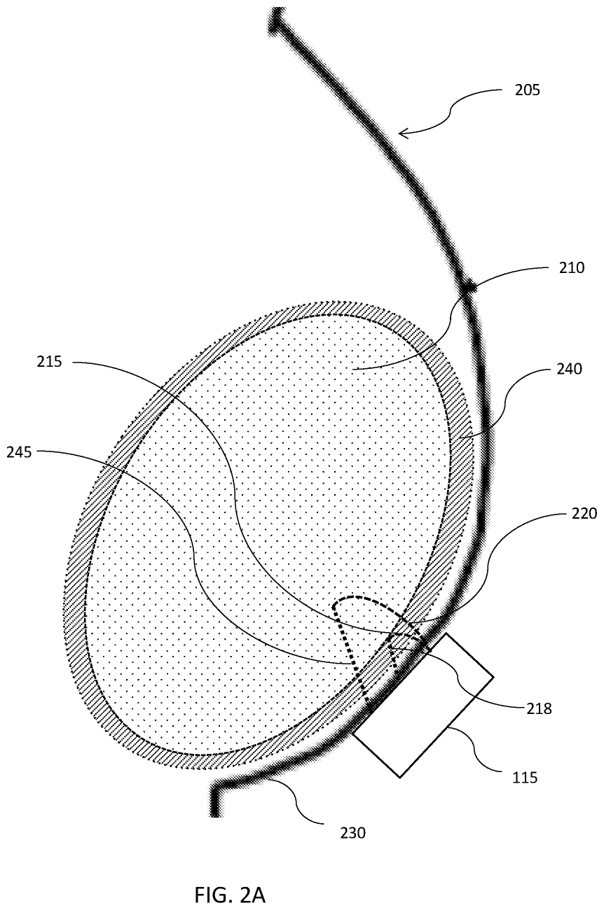

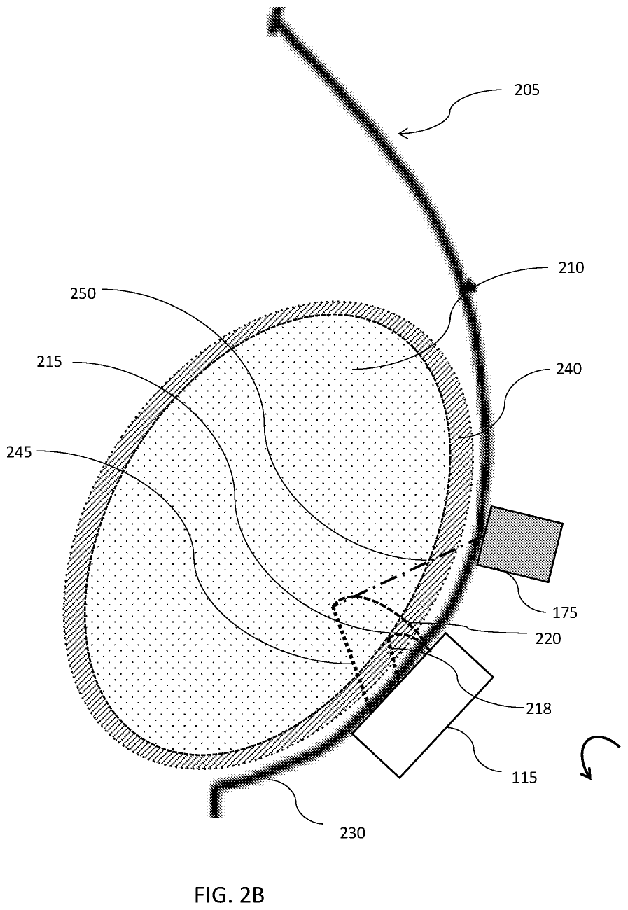

FIG. 2A illustrates provides a midsagittal plane view of pregnant mammal's 205 abdomen with fetal probe 115 positioned thereon. As shown in FIG. 2A, the pregnant mammal's abdomen 205 includes an approximation of a fetus 210, a uterus 240, and maternal tissue (e.g., skin, muscle, etc.) 230. Fetal probe 115 may be positioned anywhere on the pregnant mammal's abdomen and, in some instances, more than one fetal probe 115 may be placed on the pregnant mammal's abdomen. FIG. 2A further shows a first optical signal 215 and a second optical signal 220 being projected into the pregnant mammal's abdomen and, in the case of the second optical signal 220, fetus 210. Also shown in FIG. 2A are a first acoustic signal 218 and a second acoustic signal 245 both of which may be generated via thermoplastic expansion of the tissue and emission of soundwaves responsively to the tissue's interaction with the respective first and second optical signals 215/220 in accordance with the photoacoustic effect. Second optical signal's 220 interaction with the maternal and fetal tissue generates (via the photoacoustic effect) a composite acoustic signal 245 which may be detected by a detector like audio detector 162.

First optical signal 215 may be configured and/or projected into the pregnant mammal's abdomen so that it only penetrates the layers of the pregnant mammal's abdomen. This may be achieved by, for example, setting an intensity of first optical signal 215 and/or a source and detector distance so that first acoustic signal 218 is generated via interaction (i.e., the photoacoustic effect) between first optical signal 215 and the maternal abdominal tissue. Thus, first acoustic signal 218 may provide information about the pregnant mammal and/or the pregnant mammal's abdominal tissue, which may be helpful when trying to separate composite acoustic signal into the portion of the composite acoustic signal contributed by the pregnant mammal and the portion of the composite acoustic signal contributed by the fetus as described below.

FIG. 2B illustrates provides a midsagittal plane view of pregnant mammal's 205 abdomen with fetal probe 115 and acousto-optic modulator 177 positioned thereon. Acousto-optic modulator 177 may project a sound signal 250 into the pregnant mammal's abdomen so that, for example, the fetal and/or maternal tissue is modulated. This modulation may impact the optical signal 220 and/or composite acoustic signal 245.

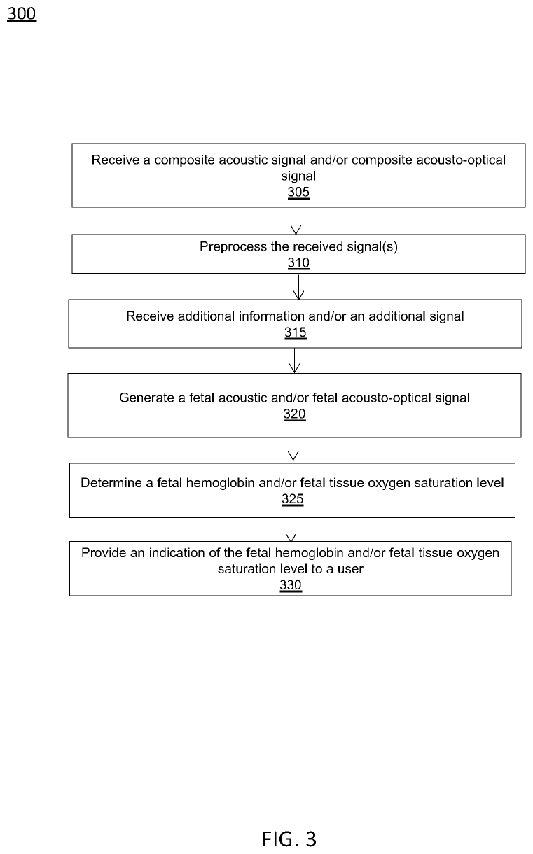

FIG. 3 provides a flowchart illustrating a process 300 for determining a fetus's tissue and/or hemoglobin oxygen saturation using a photoacoustic signal. Process 300 may be executed by, for example, system 100 and/or a component or combination of components thereof.

In step 305, a composite acoustic signal and/or a composite acousto-optical signal may be received by, for example, a computer or processor like computer 150. The composite acoustic signal may be resultant from an interaction between a pulse of light projected into the pregnant mammal's abdomen and the pregnant mammal's and/or fetus's tissue, which may be caused by the photoacoustic effect. The composite acoustic signal may be within the ultrasonic frequency range (e.g., 20 kHz-10 MHz) and may be detected by an acoustic detector (e.g., microphone and/or ultrasonic detector) like acoustic detector 162 and communicated to/received by a processor or computer like computer 150. The pulse of light may be projected by light source, such as light source 105, which may be a pulsed laser. An example of light projected into a pregnant mammal's abdomen is light beam 220 and an example, of a composite acoustic signal is composite acoustic signal 245; both of which are shown in FIGS. 2A and 2B and discussed above.

Additionally, or alternatively, the signal received in step 305 may be a composite acousto-optical signal. The composite acousto-optical signal may be resultant from an interaction between a sound signal and optical signal projected into the pregnant mammal's abdomen wherein the sound signal modulates the optical signal. The composite acousto-optical signal may be generated in a manner similar to that shown in FIG. 2B where an acousto-optic modulator like acousto-optic modulator 177 may project a sound signal like sound signal 250 into the pregnant mammal's abdomen so that, for example, the fetal and/or maternal tissue is modulated. This modulation may impact an optical signal (like optical signal 245) incident on the pregnant mammal's abdomen. In some embodiments, the acousto-optic modulator may be configured to project the sound signal into the pregnant mammal's abdomen so that it interacts with only fetal tissue that is in the path of an optical signal like optical signal 220.

Optionally, in step 310, the composite acoustic signal and/or composite acousto-optical signal may be pre-processed in order to, for example, reduce noise, amplify a desired portion of the composite acoustic/composite acousto-optical signal, and/or filter undesired portions of the composite acoustic signal/composite acousto-optical signal from the respective composite acoustic/composite acousto-optical signal. Execution of the pre-processing may include, but is not limited to, application of filtering techniques to the composite acoustic signal, application of amplification techniques the composite acoustic signal, and utilization of a lock-in amplifier on the composite acoustic signal. The filtering may, for example reduce noise or hum in the composite acoustic signal that may be caused by, for example, ambient noise, noise generated by equipment used to gather information for use with the pregnant mammal and/or noise generated by the pregnant mammal herself (e.g., breathing, heartbeat, etc.).

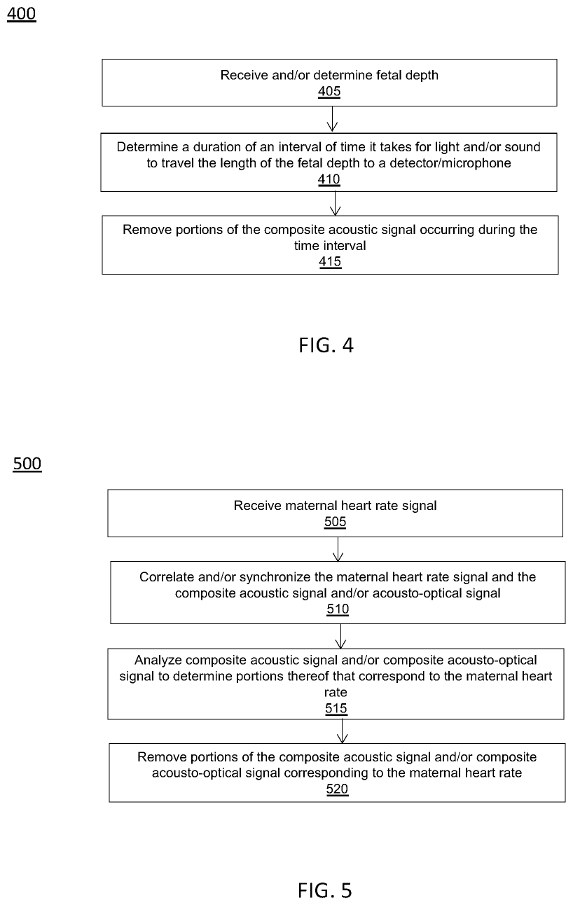

Optionally, in step 315 additional information and/or an additional signal may be received. Exemplary additional information includes, but is not limited to, a fetal depth (i.e., a distance between the pregnant mammal's epidermis and the fetus' epidermis and/or a width of the maternal abdomen), a fetal heart rate, a maternal heart rate, a noise signal, short separation information, a maternal acoustic signal, a maternal pulse oximetry signal, and/or a maternal respiratory signal. Further information regarding the additional information/signal(s) that may be received in step 315 and how it/they may be used to isolate and/or extract a portion of the composite acoustic signal that is contributed by the fetus is shown in FIGS. 4, 5, 6, and 7 and discussed below with regard to execution of one or more of process(es) 400, 500, 600, and/or 700, respectively.

The composite acoustic signal and/or composite acousto-optical signal or, when step 310 is executed, the preprocessed composite acoustic/composite acousto-optical signal, may then be processed and/or analyzed to generate a fetal acoustic signal and/or fetal composite acousto-optical signal by, for example, isolating and/or extracting a portion of the respective composite acoustic signal and/or composite acousto-optical signal that is contributed by the fetus, includes an acoustic signal generated by the fetus responsively to the light pulse incident thereon (for the composite acoustic signal), and/or includes a modulated optical signal (for the composite acousto-optical signal) (step 320). In some embodiments, the generation of a fetal acoustic signal and/or fetal composite acousto-optical signal of step 320 may be performed using additional information and/or an additional signal(s) received in step 315. The isolated and/or extracted portion of the composite acoustic signal that is contributed by the fetus may referred to herein as a “fetal acoustic signal.” The isolated and/or extracted portion of the composite acousto-optical signal that is contributed by the fetus may referred to herein as a “fetal composite acousto-optical signal.” In some embodiments, a plurality (e.g., 100, 1,000, 10,000) of composite acoustic signals and/or composite acousto-optical signals are received prior to executing step 310 (when performed) and/or step 320 and step 310 and/or 320 may be executed a plurality of times using the plurality of composite acoustic signals and/or composite acousto-optical signals, respectively and the additional information received in step 315 to generate, for example, an average, or statistically robust, fetal acoustic signal and/or fetal composite acousto-optical signal.

In some embodiments, a received composite acoustic signal and/or composite acousto-optical signal may represent a respective composite acoustic signal and/or composite acousto-optical signal that has been detected over a period of time (e.g., 1 minute, 5 minutes, etc.) and the additional information received in step 315 may be a timestamp may be present in and/or associated with the respective composite acoustic signal and/or composite acousto-optical signal. The timestamp may mark, for example, a baseline starting time (e.g., a date, time, etc.) that, in some instances, may be associated with an absolute time (e.g., chronological time) and/or a simultaneous starting point of taking a measurement (e.g., time=0). In some embodiments, a timestamp may be inserted into a signal by introducing, for example, an electrical ground, an optical signal, and/or an acoustic signal into the composite acoustic signal and the additional signal received in 315. This timestamp may enable the synchronization of the composite acoustic signal and/or composite acousto-optical signal with another signal (e.g., fetal heart rate, maternal heart rate, maternal respiratory signal, etc.) received in step 315 so that, for example, events within the composite acoustic signal and/or a fetal acoustic signal generated therefrom and/or events within the composite acousto-optical signal and/or fetal composite acousto-optical signal generated therefrom and the additional signal may correspond to one another in time.

In step 325, the isolated fetal acoustic signal and/or fetal composite acousto-optical signal may then be analyzed to determine a level of fetal hemoglobin oxygenation and/or fetal tissue oxygenation. This analysis may include generation of a two-dimensional and/or three-dimensional image of the fetus and/or pregnant mammal's abdomen where regions of the image(s) show differences in hemoglobin and/or tissue oxygenation using, for example, diffuse optical tomography. The analysis of step 325 may be performed using, for example, one or more photoacoustic equations, the Beer-Lambert law, diffuse optical tomography analysis, and/or acusto-optical equations.

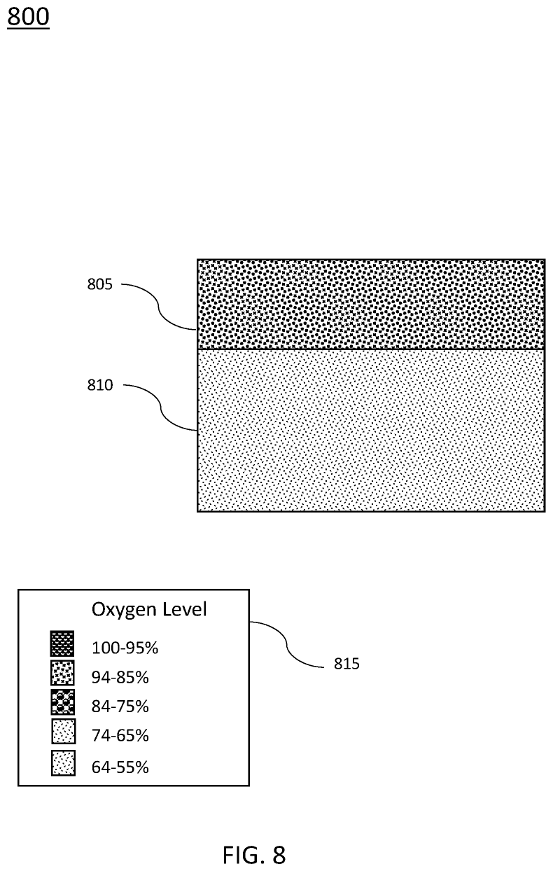

The determined level of fetal hemoglobin oxygenation and/or fetal tissue oxygenation may then be provided to a user (step 330) as, for example, an image (e.g., grey scale or color coded), a percentage, and/or a number. FIG. 8 provides an exemplary image 800 of a portion of the pregnant mammal's abdomen where a tissue oxygenation for a maternal layer 805 and a fetal layer 810 are provided as an image where a pattern within the image indicates a level of tissue oxygenation according to a key 815 provided within image 800. According to key 815, the tissue oxygenation level of the pregnant mammal is within the range of 100-95% and the tissue oxygenation level of the fetus is within the range of 74-65%.

FIG. 4 is a flowchart illustrating an exemplary process 400 for performing step 320 where the additional information received in step 315 is a fetal depth. Process 400 may be executed by, for example, system 100 and/or any component thereof.

In step 405, the additional information of a fetal depth may be received and/or determined. The fetal depth may be, for example, a distance between the pregnant mammal's epidermis and the fetus's epidermis or a width of maternal tissue at a particular location. The fetal depth may be determined using, for example, information received from a Doppler and/or ultrasound probe like Doppler/ultrasound probe 135 and/or analysis of an image (e.g., MRI or another image) of the pregnant mammal's abdomen.

In step 410, a duration of an interval of time it takes for sound energy, (i.e., the composite acoustic signal) to travel the fetal depth (i.e., from the fetus to a detector, like audio detector 162, affixed and/or proximate to the pregnant mammal's epidermis) may be determined by, for example, inputting the fetal depth and known values for the speed of sound when traveling through the maternal tissue, or an approximation thereof, into Equation 1, provided below.

s=d/t Equation 1

### Where:

s=speed of sound through maternal tissue

d=fetal depth

t=time for sound to travel the distance d

For example, if a fetal depth is 28 mm (0.028 m) and an approximation of the speed of sound through the maternal tissue is 1,540 m/s, then the time it would take for sound to travel from the fetus to the detector would be 18 microseconds (i.e., 55.5 kHz). Thus, portions of the composite acoustic signal received less than 18 microseconds after the light pulse that caused the composite acoustic signal may be filtered from the composite acoustic signal because they weren't traveling long enough to have been generated by the fetus and thus were likely generated by maternal tissue.

In some embodiments, the time it takes for light to travel the fetal depth to be incident on the fetus may also be determined in step 310 and added to the time it takes for the sound to travel the fetal depth. The time it takes for light to travel the fetal depth may be determined via Equation 1 whereby the input for v is the velocity of light through tissue, which may be determined via Equation 2, provided below:

s_{M}=c/n Equation 2

Where:
s_{M}=speed of light in maternal tissue
c=speed of light in a vacuum (299,792,458 m/s)
n=index of refraction of the maternal tissue, which is within the range of 1.3-1.4
   This speed may then be input into Equation 1 to determine to determine the time it takes for light to travel the fetal depth (i.e., to reach the fetus). This time may be added to the time it takes for the sound to travel the fetal depth to determine the time it takes for sound generated by a pulse of light to reach a detector. Using, for example, an n value equal to 1.35, the calculated value for s_{M} would have a value of 220,068,487.4 m/s and the time it would take for light to travel the fetal depth of the above example, (0.028 m) would be 0.126 nanoseconds, which in most instances is negligible to the calculations. The combined value of the time for the sound and light to travel the 0.028 m of the fetal depth is 0.180001 microseconds.

Then, in step 415, the fetal acoustic signal may be generated by removing portions of the composite acoustic signal that occur during the time interval determined in step 410 because, for example, a portion of the composite acoustic signal received during this time interval has not traveled far enough to reach the fetus and/or be generated by fetal tissue.

FIG. 5 is a flowchart illustrating an exemplary process 500 for performing step 320 where the additional information received in step 315 is a maternal heart rate signal. Process 500 may be executed by, for example, system 100 and/or any component thereof. In some embodiments, process 400 and 500 may both be executed to generate the fetal acoustic signal.

In step 505, a maternal heart rate signal may be received from, for example, a device configured to monitor or record the maternal heart rate such as an ECG device like ECG device 170, a pulse oximetry device like pulse oximetry probe 130, and/or a NIRS hemoglobin probe like NIRS adult hemoglobin probe 125.

In step 510, the maternal heart rate signal may be synchronized and/or correlated with the composite acoustic signal and/or composite acousto-optical signal. Step 510 may be executed using, for example, a timestamp present in both the maternal heart rate signal and the composite acoustic signal to correlate the maternal heart rate signal and that composite acoustic signal in time. The timestamp may represent, for example, a synchronous start time for measuring the composite acoustic signal and maternal heart rate signal and/or a chronological time when, for example, measuring of the maternal heart rate signal, composite acoustic signal, and/or composite acousto-optical signal began. The timestamp may be inserted into the maternal heart rate signal, the composite acoustic signal, and/or the composite acousto-optical signal by, for example, timestamping device 185.

The composite acoustic signal may then be analyzed to determine portions thereof that correspond to the maternal heart rate signal (step 515) so that portions of the composite acoustic signal that correspond to the maternal heart rate signal may be removed from the composite acoustic signal (step 520) to generate the fetal acoustic signal.

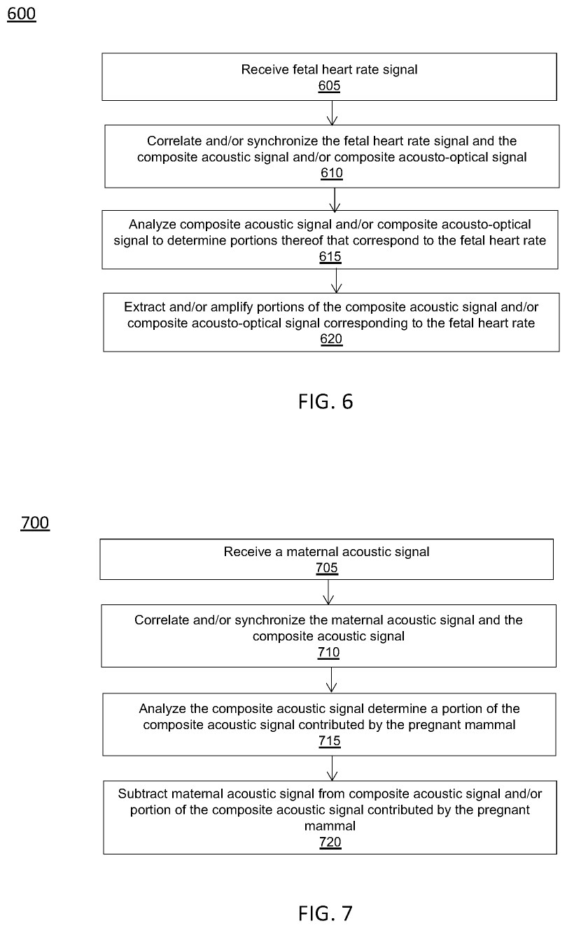

FIG. 6 is a flowchart illustrating an exemplary process 600 for performing step 320 where the additional information received in step 315 is a fetal heart rate signal. Process 600 may be executed by, for example, system 100 and/or any component thereof. In some embodiments, process 400, 500, and/or 600 may be executed, in any combination, to generate the fetal acoustic signal.

In step 605, a fetal heart rate signal may be received from, for example, a device configured to monitor or record the fetal heart rate such as an ECG device like fetal ECG device 137 and/or a Doppler and/or ultrasound device like Doppler and/or ultrasound probe 135.

In step 610, the fetal heart rate signal may be synchronized and/or correlated with the composite acoustic signal and/or composite acousto-optical signal and, when using, the maternal heart rate signal received in step 505. Step 610 may be executed using, for example, a timestamp present in the fetal heart rate signal, the composite acoustic signal, and/or composite acousto-optical signal to correlate the fetal heart rate signal and the composite acoustic signal and/or composite acousto-optical signal in time. The timestamp may represent, for example, a synchronous start time for measuring the fetal heart rate signal, composite acoustic signal, and/or composite acousto-optical signal and/or a chronological time when, for example, measuring of the fetal heart rate signal, composite acoustic signal, and/or composite acousto-optical signal began. The timestamp may be inserted into the fetal heart rate signal, the composite acoustic signal, and/or composite acousto-optical signal by, for example, timestamping device 185.

The composite acoustic signal and/or composite acousto-optical signal may then be analyzed to determine portions thereof that correspond to the fetal heart rate signal (step 615) so that portions of the composite acoustic signal and/or composite acousto-optical signal that correspond to the fetal heart rate signal may be extracted from, and/or amplified in, the respective composite acoustic signal and/or composite acousto-optical signal to generate the respective fetal acoustic signal and/or fetal composite acousto-optical signal (step 620).

FIG. 7 is a flowchart illustrating an exemplary process 700 for performing step 320 where the additional information received in step 315 is a maternal acoustic signal. Process 700 may be executed by, for example, system 100 and/or any component thereof. In some embodiments, process 400, 500, 600, and/or 700 may be executed, in any combination, to generate the fetal acoustic signal.

In step 705, a maternal acoustic signal may be received. Maternal acoustic signal may be similar to first acoustic signal 218 as described above with regard to FIG. 2A.

Optionally, in step 710, the maternal acoustic signal may be synchronized and/or correlated with the composite acoustic signal received in step 305. Step 710 may be executed using, for example, a timestamp present in the maternal acoustic signal and the composite acoustic signal to correlate the maternal acoustic signal and the composite acoustic signal in time.

In step 715, the composite acoustic signal may be analyzed to determine a portion of the composite acoustic signal contributed by the pregnant mammal and/or that corresponds with the maternal acoustic signal so that this portion of the composite acoustic signal may be subtracted the portion of the composite acoustic signal so that only the fetal acoustic signal remains (step 720).

We claim:

1. A method comprising:
receiving, by a processor, a detected composite acoustic signal that has emanated from an abdomen of a pregnant mammal, the composite acoustic signal resulting from an optical signal incident on the pregnant mammal's abdomen and a fetus contained therein;
processing, by the processor, the composite acoustic signal to isolate a portion of the composite acoustic signal that was incident on the fetus thereby generating a fetal acoustic signal;
analyzing, by the processor, the fetal acoustic signal to determine a fetal hemoglobin oxygen saturation level; and
providing, by the processor, an indication of the fetal hemoglobin oxygen saturation level to a user.

2. The method of claim 1, wherein the composite acoustic signal is caused by transient thermoelastic expansion resultant from an interaction of the pregnant mammal's abdominal tissue and the fetus' tissue to the optical signal.

3. The method of claim 1 or 2, further comprising:
preprocessing, by the processor, the composite acoustic signal prior to processing the composite acoustic signal to isolate a portion of the composite acoustic signal to remove noise from the composite acoustic signal.

4. The method of any of claims 1-3, further comprising:
receiving, by the processor, additional information prior to processing the composite acoustic signal to isolate a portion of the composite acoustic signal to remove noise from the composite acoustic signal, wherein the processing of the composite acoustic signal to isolate a portion of the composite acoustic signal to remove noise from the composite acoustic signal is performed using the additional information.

5. The method of claim 4, wherein the additional information is a fetal depth, a maternal heart rate, a fetal heart rate, a maternal hemoglobin oxygenation level, and a maternal acoustic signal.

6. The method of any of claims 1-5, further comprising:
receiving, by the processor, a maternal heart rate signal, wherein the composite acoustic signal and maternal heart rate signal include a timestamp;
synchronizing, by the processor, the maternal heart rate signal and the composite acoustic signal in time using the timestamp, wherein the processing of the composite acoustic signal to isolate a portion of the composite acoustic signal to remove noise from the composite acoustic signal comprises:
determining a portion of the composite acoustic signal that corresponds with the maternal heart rate signal; and
subtracting the portion of the composite acoustic signal that corresponds to the maternal heart rate signal from the composite acoustic signal.

7. The method of any of claims 1-6, further comprising:
receiving, by the processor, a fetal heart rate signal, wherein the composite acoustic signal and fetal heart rate signal include a timestamp;
synchronizing, by the processor, the fetal heart rate signal and the composite acoustic signal in time using the timestamp, wherein the processing of the composite acoustic signal to isolate a portion of the composite acoustic signal to remove noise from the composite acoustic signal comprises:
determining a portion of the composite acoustic signal that corresponds with the fetal heart rate signal; and
amplifying the portion of the composite acoustic signal that corresponds to the fetal heart rate signal from the composite acoustic signal.

8. The method of any of claims 1-7, further comprising:
receiving, by the processor, a fetal heart rate signal, wherein the composite acoustic signal and fetal heart rate signal include a timestamp;
synchronizing, by the processor, the fetal heart rate signal and the composite acoustic signal in time using the timestamp, wherein the processing of the composite acoustic signal to isolate a portion of the composite acoustic signal to remove noise from the composite acoustic signal comprises:
determining a portion of the composite acoustic signal that corresponds with the fetal heart rate signal; and
extracting the portion of the composite acoustic signal that corresponds to the fetal heart rate signal from the composite acoustic signal.

9. The method of any of claims 1-8, further comprising:
receiving, by the processor, a fetal depth for the fetus within the pregnant mammal's abdomen; and
determining, by the processor, a duration of an interval of time it takes for sound to travel the length of the fetal depth to a detector, wherein the processing of the composite acoustic signal to isolate a portion of the composite acoustic signal comprises:
removing, by the processor, portions of the composite acoustic signal that occur during the interval of time.

10. The method of any of claims 1-9, wherein the processor controls operation of a source of the optical signal, the method further comprising:
receiving, by the processor, a fetal depth for the fetus within the pregnant mammal's abdomen, wherein an operation of the source of the optical signal is adjusted responsively to the fetal depth.

11. The method of any of claims 1-10, wherein the processor controls operation of a source of the optical signal, the method further comprising:
determining, by the processor, whether the composite acoustic signal is of sufficient strength; and
adjusting, by the processor, an operation of the source of the optical signal responsively to a determination that the composite acoustic signal is not of sufficient strength.

12. The method of any of claims 1-11, wherein the processor controls operation of a source of the optical signal, the method further comprising:
determining, by the processor, whether the fetal acoustic signal is of sufficient strength; and
adjusting, by the processor, an operation of the source of the optical signal responsively to a determination that the fetal acoustic signal is not of sufficient strength.

13. The method of any of claims 1-12, further comprising:
receiving, by the processor, a maternal acoustic signal, wherein isolation of the fetal acoustic signal comprises:
determining a portion of the composite acoustic signal that corresponds with the maternal acoustic signal; and
subtracting the portion of the composite acoustic signal that corresponds to the maternal acoustic signal from the composite acoustic signal.

14. The method of any of claims 1-13, wherein the composite acoustic signal is timestamped, the method further comprising:
receiving, by the processor, a timestamped maternal acoustic signal, wherein isolation of the fetal acoustic signal comprises:
synchronizing the timestamped composite acoustic signal and maternal acoustic signal using a timestamp present in the composite acoustic signal and a timestamp present in the maternal acoustic signal;
determining a portion of the composite acoustic signal that corresponds with the maternal acoustic signal; and
subtracting the portion of the composite acoustic signal that corresponds to the maternal acoustic signal from the composite acoustic signal.

15. A method comprising:
receiving, by a processor, a detected composite acoustic signal that has emanated from an abdomen of a pregnant mammal, the composite acoustic signal resulting from an optical signal incident on the pregnant mammal's abdomen and a fetus contained therein;
processing, by the processor, the composite acoustic signal to isolate a portion of the composite acoustic signal that was incident on the fetus thereby generating a fetal acoustic signal;
analyzing, by the processor, the fetal acoustic signal to determine a fetal tissue oxygen saturation level; and
providing, by the processor, an indication of the fetal tissue oxygen saturation level to a user.

16. The method of claim 15, wherein the composite acoustic signal is caused by transient thermoelastic expansion resultant from an interaction of the pregnant mammal's abdominal tissue and the fetus' tissue to the optical signal.

17. The method of claim 15 or 16, further comprising:
preprocessing, by the processor, the composite acoustic signal prior to processing the composite acoustic signal to isolate a portion of the composite acoustic signal to remove noise from the composite acoustic signal.

18. The method of any of claims 15-17, further comprising:
receiving, by the processor, additional information prior to processing the composite acoustic signal to isolate a portion of the composite acoustic signal to remove noise from the composite acoustic signal, wherein the processing of the composite acoustic signal to isolate a portion of the composite acoustic signal to remove noise from the composite acoustic signal is performed using the additional information.

19. The method of claim 18, wherein the additional information is a fetal depth, a maternal heart rate, a fetal heart rate, a maternal hemoglobin oxygenation level, and a maternal acoustic signal.

20. The method of any of claims 15-19, further comprising:
receiving, by the processor, a maternal heart rate signal, wherein the composite acoustic signal and maternal heart rate signal include a timestamp;
synchronizing, by the processor, the maternal heart rate signal and the composite acoustic signal in time using the timestamp, wherein the processing of the composite acoustic signal to isolate a portion of the composite acoustic signal to remove noise from the composite acoustic signal comprises:
determining a portion of the composite acoustic signal that corresponds with the maternal heart rate signal; and
subtracting the portion of the composite acoustic signal that corresponds to the maternal heart rate signal from the composite acoustic signal.

21. The method of any of claims 15-20, further comprising:
receiving, by the processor, a fetal heart rate signal, wherein the composite acoustic signal and fetal heart rate signal include a timestamp;
synchronizing, by the processor, the fetal heart rate signal and the composite acoustic signal in time using the timestamp, wherein the processing of the composite acoustic signal to isolate a portion of the composite acoustic signal to remove noise from the composite acoustic signal comprises:
determining a portion of the composite acoustic signal that corresponds with the fetal heart rate signal; and
amplifying the portion of the composite acoustic signal that corresponds to the fetal heart rate signal from the composite acoustic signal.

22. The method of any of claims 15-21, further comprising:
receiving, by the processor, a fetal heart rate signal, wherein the composite acoustic signal and fetal heart rate signal include a timestamp;
synchronizing, by the processor, the fetal heart rate signal and the composite acoustic signal in time using the timestamp, wherein the processing of the composite acoustic signal to isolate a portion of the composite acoustic signal to remove noise from the composite acoustic signal comprises:
determining a portion of the composite acoustic signal that corresponds with the fetal heart rate signal; and
extracting the portion of the composite acoustic signal that corresponds to the fetal heart rate signal from the composite acoustic signal.

23. The method of any of claims 15-22, further comprising:
receiving, by the processor, a fetal depth for the fetus within the pregnant mammal's abdomen; and
determining, by the processor, a duration of an interval of time it takes for sound to travel the length of the fetal depth to a detector, wherein the processing of the composite acoustic signal to isolate a portion of the composite acoustic signal comprises:
removing, by the processor, portions of the composite acoustic signal that occur during the interval of time.

24. The method of any of claims 15-23, wherein the processor controls operation of a source of the optical signal, the method further comprising:
receiving, by the processor, a fetal depth for the fetus within the pregnant mammal's abdomen, wherein an operation of the source of the optical signal is adjusted responsively to the fetal depth.

25. The method of any of claims 15-24, wherein the processor controls operation of a source of the optical signal, the method further comprising:
determining, by the processor, whether the composite acoustic signal is of sufficient strength; and
adjusting, by the processor, an operation of the source of the optical signal responsively to a determination that the composite acoustic signal is not of sufficient strength.

26. The method of any of claims 15-25, wherein the processor controls operation of a source of the optical signal, the method further comprising:
determining, by the processor, whether the fetal acoustic signal is of sufficient strength; and
adjusting, by the processor, an operation of the source of the optical signal responsively to a determination that the fetal acoustic signal is not of sufficient strength.

27. The method of any of claims 15-26, further comprising:
receiving, by the processor, a maternal acoustic signal, wherein isolation of the fetal acoustic signal comprises:
determining a portion of the composite acoustic signal that corresponds with the maternal acoustic signal; and
subtracting the portion of the composite acoustic signal that corresponds to the maternal acoustic signal from the composite acoustic signal.

28. The method of any of claims 15-27, wherein the composite acoustic signal is timestamped, the method further comprising:
receiving, by the processor, a timestamped maternal acoustic signal, wherein isolation of the fetal acoustic signal comprises:
synchronizing the timestamped composite acoustic signal and maternal acoustic signal using a timestamp present in the composite acoustic signal and a timestamp present in the maternal acoustic signal;
determining a portion of the composite acoustic signal that corresponds with the maternal acoustic signal; and
subtracting the portion of the composite acoustic signal that corresponds to the maternal acoustic signal from the composite acoustic signal.
